(19) United States
(12) Patent Application Publication
(10) Pub. No.: US 2007/0054347 A1
(43) Pub. Date: Mar. 8, 2007

(54) Oxidative stress measurement device and related methods

(75) Inventors: Scott Rosendahl, Montreal (CA); Dirk Bandilla, Montreal (CA); Hyman Schipper, Montreal (CA); David H. Burns, Montreal (CA)

Correspondence Address: BERESKIN AND PARR, 40 KING STREET WEST, BOX 401, TORONTO, ON, M5H 3Y2, CA

(73) Assignee: McGILL UNIVERSITY, Montreal (CA)

(21) Appl. No.: 11/357,060

(22) Filed: Feb. 21, 2006

Related U.S. Application Data
(60) Provisional application No. 60/654,497, filed on Feb. 22, 2005.

(51) Int. Cl. C12Q 1/26; G06F 19/00; A61B 5/00

(52) U.S. Cl. 435/25; 600/315; 702/19

(57) ABSTRACT

For measuring an oxidative stress component in a patient, an optical analyzer having a light source and a light detector is used for measuring an optical property of a medium and generating optical measurement data. A processor analyzes the optical measurement data and generates a value for one or more oxidative stress component in the form of a redox signature for the patient. Probability data of the presence of an oxidative stress dependent disease can be calculated. By observing at least one additional clinical condition of the disease, a diagnosis using said at least one additional condition and said redox signature can be obtained.

### CROSS-REFERENCE TO RELATED APPLICATIONS

This application claim priority of U.S. provisional patent application 60/654,497 filed Feb. 22, 2005.

### TECHNICAL FIELD

This invention relates in general to the field of measurement of oxidative stress in biological systems, and also to the field of detecting or diagnosing Alzheimer's disease, Parkinson's disease and other diseases associated with oxidative stress products in biological fluids. The invention also relates to the study of drug efficacy.

### BACKGROUND OF THE INVENTION

Free radicals are atoms or molecules that contain unpaired electrons in their outer orbitals. Their electronic configurations render these chemical species highly reactive with membrane lipids, proteins, nucleic acids and other cellular substrates. Free radicals may be derived from environmental sources or may be generated de novo within tissues. The superoxide anion (O2-), hydrogen peroxide (H2O2), singlet oxygen, hypochlorous acid (HOCl), peroxynitrite (ONOO—) and the hydroxyl radical (OH) are examples of common, endogenously-produced reactive oxygen species (ROS). Transition metals, such as ferrous iron (Fe2+) or cuprous copper (Cu1+), play a vital role in cellular redox chemistry by reducing H2O2 to the highly-cytotoxic OH radical (Fenton catalysis). In mammalian tissues, evolutionarily-conserved antioxidant enzymes (e.g. the superoxide dismutases, catalase, the glutathione peroxidases and various reductases) operate in concert with a host of non-enzymatic, low-molecular-weight antioxidant compounds (e.g. GSH, thioredoxin, ascorbate, the tocopherols, uric acid, melatonin, bilirubin) to maintain redox homeostasis. By maintaining transition metals in a relatively low redox state, metal-binding proteins, such as ferritin, transferrin, lactoferrin, the metallothioneins and ceruloplasmin, contribute substantially to the antioxidant protection of tissues and biological fluids.

Oxidative stress (OS) has been defined as “a disturbance in the pro-oxidant/antioxidant balance in favor of the former, leading to possible [tissue] damage” [Sies, H., Oxidative Stress. Oxidants and Antioxidants. 1991, New York: Elsevier. 507]. This balance can be related to one or more biochemical component of the biological fluid. Oxidative stress has been implicated as a key common pathway for cellular dysfunction and death and a potential therapeutic target in a broad spectrum of human medical conditions including cancer, diabetes, obstructive lung disease, inflammatory bowel disease, cardiac ischemia, glomerulonephritis, macular degeneration and various neurodegenerative disorders [Halliwell, B. and J. M. C. Gutteridge, Free Radicals in Biology and Medicine. 3 ed. 1999, Oxford: Oxford University Press Inc. 736].

Oxidative Stress in AD: Alzheimer's disease (AD) is a dementing illness characterized by progressive neuronal degeneration and the accumulation of intracellular inclusions (neurofibrillary tangles) and extracellular deposits of amyloid (senile plaques) in discrete regions of the basal forebrain, hippocampus, and association cortices [Selkoe, D. J., The molecular pathology of Alzheimer's disease. Neuron, 1991. 6(4): p. 487-98]. Oxidative stress (OS) has been consistently implicated in the pathogenesis of this condition [Reichmann, H. and P. Riederer, Mitochondrial disturbances in neurodegeneration. Neurodegenerative Diseases, ed. D. B. Calne. 1994, Philadelphia: Saunders. 195-204; Beal, M., Mitochondrial Dysfunction and Oxidative Damage in Neurodegenerative Diseases. 1995: Landes, R. G. 1-128; Mattson, M. P., Contributions of mitochondrial alterations, resulting from bad genes and a hostile environment, to the pathogenesis of Alzheimer's disease. Int Rev Neurobiol, 2002. 53: p. 387-409]. OS in AD brain is evidenced by (i) mitochondrial insufficiency, which is both a cause and consequence of free radical generation in injured tissues, (ii) augmented levels of oxidatively-modified lipid, protein and nucleic acids relative to age-matched non-demented subjects, (iii) perturbations of antioxidant enzyme concentrations and activities, (iv) increased deposition of iron and other redox-active transition metals, and (v) purported attenuation of disease progression following therapeutic administration of antioxidants, alpha-tocopherol (vitamin E), Ginkgo biloba extract, and N-acetylcysteine [Schipper, H. M., Redox neurology: visions of an emerging subspecialty. Ann N Y Acad Sci, 2004. 1012: p. 342-55]. In recent years, it has become apparent that OS in AD patients is not limited to affected brain tissue but occurs, and can be detected and quantified, in cerebrospinal fluid (CSF) and the systemic circulation. For example, augmented levels of isoprostanes, 8-OHdG and protein carbonyls, biochemical indices of lipid, nucleic acid and protein oxidation, and abnormal antioxidant enzyme activities have been repeatedly documented in the CSF, blood plasma and blood serum of patients with early and advanced AD and, in some cases, in subjects with Mild Cognitive Impairment (MCI) [Yu, H.-L., et al., Aberrant profiles of native and oxidized glycoproteins in Alzheimer plasma. Proteomics, 2003. 3: p. 2240-2248])

Using 2-dimensional gel electrophoresis and mass spectrometry, specific proteins particularly susceptible to OS-related modification have been isolated and identified in brain homogenates, CSF and blood of AD patients [Yu, H.-L., et al., Aberrant profiles of native and oxidized glycoproteins in Alzheimer plasma. Proteomics, 2003. 3: p. 2240-2248].

Biological Markers of Sporadic AD: “Sporadic Alzheimer's Disease” refers to AD in a patient with no predisposition to AD due to family history. Although several candidate biomarkers of sporadic AD have been identified and commercialized, none currently fulfills criteria enabling an ideal test (Neurobiology of Aging 19:107-167, 1998), namely one based on rapid non-invasive sampling and yielding objective data of high specificity and sensitivity. Several laboratories have demonstrated abnormally low levels of A 1-42 and increased concentrations of total Tau, phospho-Tau and neural thread protein in the CSF of sporadic AD patients.

Although tests are known which can differentiate between a normal elderly control (NEC) individual and an individual affected by AD, they suffer from significant limitations. For example, combined measurements of CSF A 1-42 and phospho-Tau discriminate early AD from NEC and other dementing conditions with sensitivities and specificities in the range of 80-85% (ref). However, CSF examination by lumbar puncture is relatively invasive and therefore not suitable for mass screening of elderly individuals with AD risk factors or mild memory impairment [Galasko, D., New approaches to diagnose and treat Alzheimer's disease: a glimpse of the future. Clin Geriatr Med, 2001. 17(2): p. 393-410; Ferrarese, C. and M. Di Luca, Biological markers in Alzheimer's disease. Neurobiol Aging, 2003. 24(1): p. 191-3]. In 1996, Kennard and co-workers reported increased serum levels of the iron-binding protein, p97 (melanotransferrin) in Canadian and Japanese patients with sporadic AD. However, the degree of overlap between AD and control subjects was subsequently determined to be greater than initially surmised. Moreover, a recent study has called into question the specificity of the antisera used to measure the p97 protein [Desrosiers, R. R., et al., Expression of melanotransferrin isoforms in human serum: relevance to Alzheimer's disease. Biochem J, 2003. 374(Pt 2): p. 463-71]. Measurement of elevated AD7C-neural thread protein in urine purportedly differentiates AD from control subjects with high sensitivity and specificity. However, urinary AD7C levels are extremely low (means for AD and control subjects are 2.5 and 0.8 ng/ml, respectively) requiring extensive protein purification procedures prior to immunoassay [Ghanbari, H., et al., Biochemical assay for AD7C-NTP in urine as an Alzheimer's disease marker. J Clin Lab Anal, 1998. 12(5): p. 285-8].

It is thus known to measure oxidative stress in blood plasma and cerebrospinal fluid (CSF) using chromatographic techniques and mass spectrometry. Such analytical techniques are time consuming and typically involve obtaining a significant quantity of biological fluid to obtain a measurement of oxidative stress.

In this specification, the term “biological fluid” is intended to mean, without limitation, whole blood, blood plasma, blood serum, urine, saliva, tear fluid, cerebrospinal fluid (CSF), amniotic fluid and breath.

In this specification, the term “patient” is intended to mean a subject to be investigated, observed, monitored or studied, whether human or animal.

In this specification, the term “non-invasive” is intended to include transdermal, transcutaneous spectroscopy, or across the vaginal wall into the amniotic cavity, and minimally invasive, such as by withdrawing a small volume of biological fluid.

In this specification, the term “oxidative stress related disease” is intended to mean any disease that either causes oxidative stress or is caused by or dependent on oxidative stress.

In this specification, the term “oxidative stress component” is intended to mean the disturbance in the pro-oxidant/antioxidant balance of a biochemical component of biological fluid in favor of the former, leading to possible tissue damage”. Likewise “oxidative stress components” is intended to mean such disturbance in the pro-oxidant/antioxidant balance of a plurality of biochemical components of the biological fluid in favor of the former, leading to possible tissue damage. The term “redox signature” is intended to mean an aggregate of oxidative stress components or OS biological byproducts derived from multi-wavelength optical absorption spectroscopy or NMR spectroscopy.

### SUMMARY OF THE INVENTION

The present invention provides a system and method for optically measuring oxidative stress in biological fluids.

The present invention provides a method and apparatus for correlating spectra, such as multi-wavelength optical absorption, Raman scattering spectra, or magnetic resonance spectra, of biological fluids with an oxidative stress dependent disease.

It is not known in the art to use oxidative stress measurement in a clinical environment as a tool in diagnosing or predicting the onset of disease. The present invention provides a tool that allows rapid measurement of oxidative stress suitable for use in a clinical setting.

It is not known in the art to measure oxidative stress longitudinally (over time) with a view to better study pharmacokinetics. The present invention provides a device that is able to measure oxidative stress quickly and non-invasively in a manner suitable for use with small or large patients.

It is not known in the art to measure oxidative stress longitudinally with a view to detect onset of infection in closely monitored patients, such as those who are in critical condition or in intensive care. The present invention provides a tool that allows continuous measurement of oxidative stress suitable for use in a critical care facility.

According to a first aspect of the present invention, there is provided a tool to measure one or more oxidative stress components in biological fluid using optical analysis. The biological fluid analyzed according to the invention may be any one of, or a combination of, whole blood, blood plasma, blood serum, urine, saliva, tear fluid and cerebrospinal fluid (CSF). The optical analysis may be done with wavelengths from optical spectra in a variety of ranges, such as the NIR, SWNIR, and THz ranges. In addition to absorption spectra, Raman spectra and fluorescence spectra may also be analyzed. Nuclear Magnetic Resonance (NMR) spectroscopy may also be used to determine AD.

According to a second aspect of the present invention, there is provided a method and apparatus to determine probability data of the presence of an oxidative stress dependent disease in a patient. A correlation is established between an oxidative stress dependent disease and spectra of a biological fluid obtained using a chosen analytical modality for a population of patients. For the patient whose probability data is to be determined, a spectrum of the biological fluid is obtained using the chosen modality. The probability data for the patient is generated using the acquired spectrum and the established correlation.

According to a third aspect of the present invention, there is provided a method of clinical diagnosis of a patient, in which a measurement is obtained of one or more oxidative stress component in a biological fluid of the patient, and at least one additional condition of the patient is observed. A diagnosis of the patient is then concluded using the at least one additional condition and the oxidative stress component measurement, wherein the diagnosis is not enabled by only one of the measurement and the at least one additional condition.

According to a fourth aspect of the present invention there is provided a method of studying in a patient efficacy of a drug or treatment intended to treat an oxidative stress related disease. The method involves administering the drug or treatment to the patient, and measuring over time at least one oxidative stress component.

According to a fifth aspect of the present invention, there is provided a method of monitoring a patient in intensive care. The method involves continuously or frequently measuring at least one oxidative stress component in a patient, and detecting a change in the oxidative stress component in the patient.

### BRIEF DESCRIPTION OF THE DRAWINGS

Further features and advantages of the present invention will become apparent from the following detailed description, taken in combination with the appended drawings, in which:

FIG. 1 is a plot showing absorption spectra in the 600 nm to 1100 nm range illustrating the typical characteristics of the spectra for a normal elderly control (NEC) patient and for an Alzheimer's patient;

FIG. 2 is a schematic drawing of an optical oxidative stress measurement device having a 50 μL sample cell with a 1 cm path length supplied with light from a broadband Tungsten Halogen lamp via a first optical fiber, a short wavelength near infrared (SWNIR) spectrophotometer coupled to an opposite end of the sample cell via a second optical fiber for detecting CW intensity in the 600 nm to 1100 nm range, and a computer connected to the spectrophotometer for recording and analyzing the spectra;

FIG. 3 is graph showing absorption levels from a variety of molecular species in the 600 to 1000 nm wavelength range;

FIG. 4a is a graph comparing NEC to AD samples;

FIG. 4b is a graph comparing MCI to AD samples;

FIG. 5a is a graph comparing NEC to MCI samples;

FIG. 5b is a graph comparing NEC to VCI samples;

FIG. 6a is a graph comparing NEC to PD samples;

FIG. 6b is a graph comparing AD to PD samples;

FIG. 7 is a graph showing Raman spectrum counts for PD and NEC showing COOH, CH, C═C, NH and R—OH spectral components; and

FIG. 8 is a schematic diagram of an optical oxidative stress measurement device arranged in a reflective mode for transdermal use.

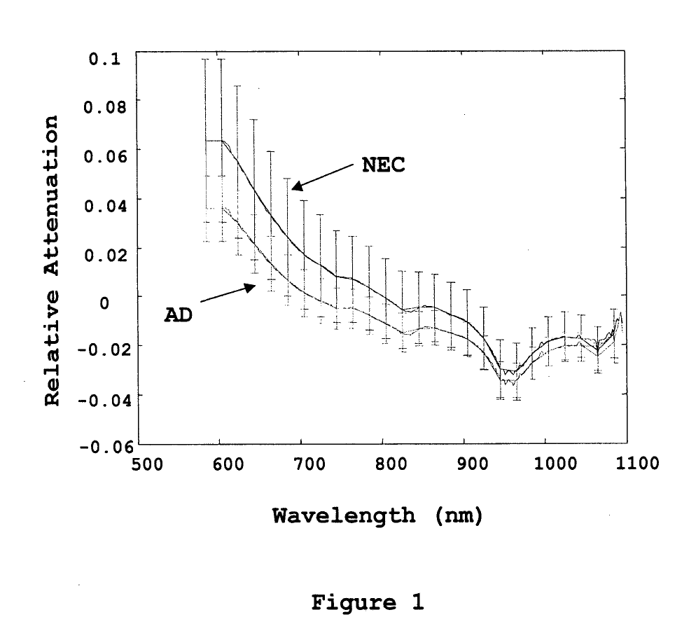

It will be noted that throughout the appended drawings, like features are identified by like reference numerals.

### DETAILED DESCRIPTION OF THE PREFERRED EMBODIMENT

Methodology

Measurement of OS from Short Wavelength Near Infrared (SWNIR) Spectroscopy of Blood Plasma

Sampling Methodology

According to prior anamesis and testing, patients were grouped into the following categories:
Normal Elderly Control (NEC)
Mild Cognitive Impairment (MCI)
Alzheimer's Disease (AD)
Vascular Cognitive Impairment (VCI)
Parkinson's disease (PD)

The patient population varied in terms of gender, age, medication, nutrition, other diseases (if any), and familial predisposition.

Initial experiments had shown that the nature of the anticoagulant (EDTA versus Heparin), variations in freezing and thawing cycles (storage at −80° C. versus +4° C.), centrifugation time (1 min versus 30 min) and preconditioning of the sample cell all had significant impact on the spectral signature of the plasma sample. Consequently, the ensuing sample preparation procedure was developed:

Blood samples were drawn from the patients and, upon centrifugation, the separated plasma immediately stored at −80° C. using either EDTA or Heparin as an anticoagulant. Storage time varied from 1 to 400 days. Only classes comprising the same anticoagulant were compared to one another.

Prior to analysis samples were thawed for one hour to reach room temperature and then centrifuged for 30 min. For cleaning and preconditioning, the sample cell was first rinsed with 200 μl of 0.1 M NaOH followed by 3×200 μl Millipore water.

Short Wavelength Near Infrared (SWNIR) Spectroscopy

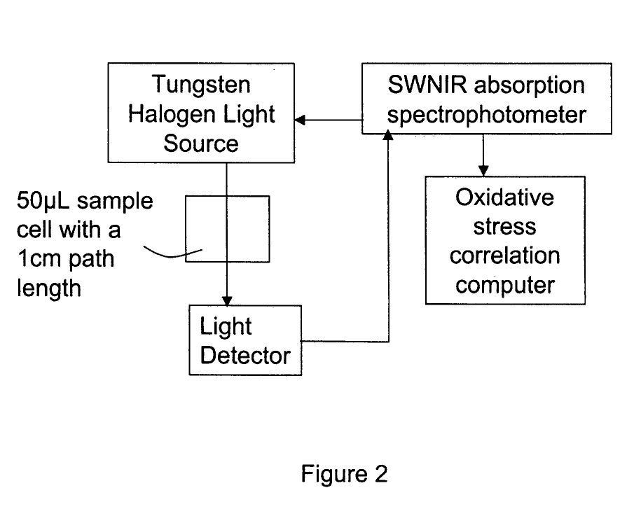

A SWNIR spectrum was recorded of the third water rinse serving as a control. Thereupon, 75 μl of sample was injected into the sample cell and a sample spectrum recorded using the apparatus as shown in FIG. 2.

Short wavelength near infrared spectra were obtained from the prepared plasma samples using the following protocol. For the measurements, an American Holographic near infrared spectrophotometer was used. The spectrophotometer is equipped with a two channel input port so that a reference could be obtained simultaneous with the measurement sample. Spectra acquired covered the 580 to 1100 nm region. Integration time of the detector was 100 milliseconds. All samples were measured 50 times and the results averaged to reduce spectral noise. Samples were introduced into a sample cell with 10 mm internal pathlength using an eppendorff pipette. Approximately 75 microliter sample was used. After spectral data were obtained, the sample cell was washed using 200 microliter-0.1 M NaOH followed by 3 volumes of 200 microliter Millipore water. After each sample a separate reference spectrum was taken of the third water rinse solution. This allowed monitoring of contamination of the sample cell or changes in alignment of the optical system. Each sample spectrum was referenced to the consecutive water sample for later processing.

Analysis Methodology

Two different methods of spectral analysis were used to identify and categorize plasma samples into neurological classes. Method one used pre-selection of wavelength regions associated with functional groups identifiable by NIR spectroscopy. Linear models of class were then determined using all possible combinations of the wavelength regions selected. In method two, the best combination of wavelength regions were automatically selected using a Haar transform based technique of the acquired SWNIR spectra at 256 wavelengths spanning 600-1100 nm. Classification of samples from SWNIR spectra consisted of determining the most parsimonious combination of variables in the wavelet domain using a genetic algorithm (GA). To obtain data in the wavelet domain “son” Haar wavelets were used. A more detailed overview of the data processing steps is given in the following sections.

A Priori Wavelength Selection

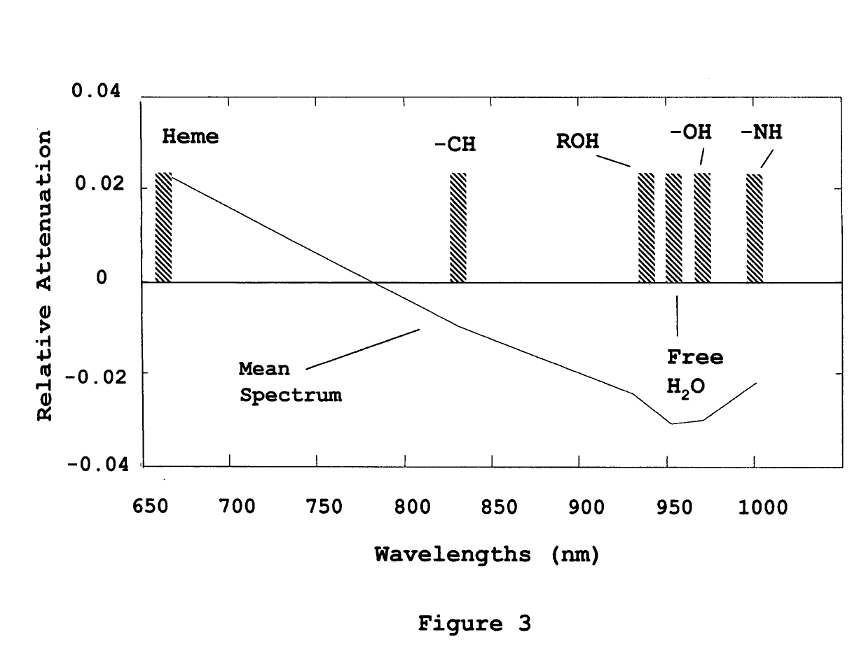

The SWNIR spectra contains absorptions from a variety of molecular species. To select for major molecular species present in blood plasma, wavelength regions (15 nm width) associated with Heme (700 nm), CH (830 nm), ROH(940 nm), H₂O (960 nm), OH(980 nm) and NH (1020 nm) moieties were identified as shown in FIG. 3. The integrated absorptions from these six regions were then used in the regression model described below.

Haar Wavelet Transform

The Haar transform (HT) is the oldest and simplest wavelet transform. Similarly to the Fourier transform, it projects data—for example a NIR spectrum—onto a given basis set. Unlike the Fourier transform, which uses sine and cosine functions as a basis set, the HT uses Haar wavelets. In this study, a discrete wavelet transform (DWT) was chosen over a continuous wavelet transform or a wavelet packet transform to maximize the simplicity and speed of calculations. For data defined over the range 0≦x≦1, the family of Haar wavelets for a DWT is given by:

$$\begin{matrix}ϕ ( x ) = \{ \begin{matrix}1 & if 0 \leq x < 1 \\ 0 & otherwise\end{matrix} & ( 1 ) \\ ø ( x ) = \{ \begin{matrix}1 & if 0 \leq x < \frac{1}{2} \\ - 1 & if \frac{1}{2} \leq x < 1 \\ 0 & otherwise\end{matrix} & ( 2 )\end{matrix}$$

ψ_{n,k}(x)=ψ(2ⁿx−k), 0≦k≦2ⁿ−1 (3)

Carrying out a HT consists of decomposing a spectrum into a weighted sum of f, y, and yn, k, where the weightings are known as “wavelet coefficients”. To obtain the coefficient of the father wavelet, f, the signal over the entire data window is integrated. The weighting of the mother wavelet, y, is obtained by integrating the first half of the data points, and subtracting the sum of the second half of the data. Daughter wavelets are scaled down and translated versions of the mother wavelet. In the notation yn, k, n represents the scaling, and k indicates translation. Thus, ever smaller regions in the data are summed to find the coefficients of the daughter wavelets, down to the minimum element size of the data. Daughter wavelets therefore behave like high-pass filters, while the father wavelet functions as a low-pass filter. Overall, the number of wavelet coefficients obtained is the same as the number of points in the original data set.

From the above description, it becomes apparent that Haar wavelets are simple structures, since they only have three distinct levels: +1, −1 or 0. It is however possible to simplify them even further by introducing scaled down and shifted versions of the father wavelet called scaling functions or “son” wavelets:
φ_{n,k}(x)=φ(2ⁿx−k), 0≦k≦2ⁿ−1 and 0≦x≦1 (4)

The mother wavelet can then be rewritten as
ψ=φ_{1,0}−φ_{1,1} (5)

A “son” HT can therefore be carried out on a spectrum with z points using 2z-1 wavelets that are constructed only of ones and zeros. The basis set for this wavelet transform is not orthogonal, since higher generation son wavelets are subsets of lower generations. However, son wavelets have the advantage of being monodirectional, i.e., they only go positive. Thus, unlike daughter wavelets, son wavelets do not inherently carry out a first derivative in the data processing.

In the context of spectral analysis, the HT is particularly well suited. Wavelet coefficients obtained contain both frequency and wavelength information (where “frequency” is not used in the usual sense, but refers to whether wavelets describe small- and large-scale features). Due to the retention of wavelength information, it is easier to understand the spectral meaning of HT results than FT results. Furthermore, it becomes possible to not only investigate the importance of separate wavelengths, but also spectral features of different sizes one common application of this property is to smooth data by deleting high frequency wavelet coefficients. Alternately, large trends in data sets such as sloping baselines can be corrected by removing low frequency wavelets. [Absi, E., et al., Decrease of 1-methyl-1,2,3,4-tetrahydroisoquinoline synthesizing enzyme activity in the brain areas of aged rat. Brain Res, 2002. 955(1-2): p. 161-3]

Another important trait of the HT is its ability to compress a large mount of information into a very small number of variables. Daughter wavelets are efficient in data compression, and this property is exploited in the present study to find the most parsimonious model to estimate sample properties. In comparison, the son HT does not perform as well for data compression since it is partially redundant, but it allows complete decoupling of adjacent wavelengths. Therefore this basis set should allow more freedom in feature selection. Furthermore, models built with son wavelets are easier to interpret. Since n, k have only two discrete levels, either a wavelength region is chosen or not chosen by the optimization algorithm. Based on the selected son wavelets, it should be possible to build a simplified instrument that uses slits or filters for sample analysis.

Both the daughter and son HT were calculated using programs written in Matlab (The MathWorks Inc., Natick, Mass.). For the daughter HT, a fast HT program based on Mallat's pyramid algorithm determined the wavelet coefficients by carrying out a series of recursive sums and differences [C. E. W. Gributs, D. H. Burns, Applied Optics, 2003, 42/16, p.2923-2930]. For the son HT, simple sums were used. Since the algorithms required the length of input data to be a power of 2, experimental spectra were padded with the last data value to reach the nearest 2n. Wavelet coefficients were determined and ordered from wide to more compact wavelets (f, y, y1, 0, . . . , yn, k or f, f1, 0, . . . , fn, k).

Genetic Algorithm

The most parsimonious subset of variables (either wavelengths from method 1 or wavelets from method 2) to estimate a class of interest was determined by inverse least squares (ILS) regression. When few wavelengths were involved, as in method 1, all possible combinations were modeled. When many wavelets were included in the classification (i.e. method 2), a genetic algorithm (GA) optimization to determine the best choice of wavelets was used. Using principles such as mating, crossover and mutation, many models were evaluated. For each variable combination, sample class were estimated according to
Y=α₀+α₁X₁+α₂X₂+ . . . +aαₙXₙ (6)
where Y is the dependent variable (neurological class, i.e. 0-normal, 1-AD), X₁, X₂, . . . , Xₙ are independent variables (i.e., intensity of a given wavelength or wavelet coefficients), and α₀, α₁, . . . , αₙ are the coefficients determined from a set of calibration X's. Complete descriptions of GA optimization have previously been given elsewhere [M. J. McShane, B. D. Cameron, G. L. Cote, C. H. Spiegelman, Applied Spectroscopy 1999, 53, p.1575-1581] therefore only an overview of the method will be given here.

The best fit (optimal) models containing 1 to 15 variables were sought using the GA method. A population of individuals (i.e., models) was created by encoding chosen variables in binary and lexicographically stacking them. For preprocessed spectra of 2n variables, the binary encoding used n bits. Population size was set to 1000. Every individual was used with a calibration set to build a model according to Equation 6. Computation of the corresponding standard error of calibration (SEC) was based on a test set. The two fittest individuals were identified based on their SEC, and kept for the next generation without mutation. The rest of the new population was filled by randomly mating individuals with a crossover probability of 1 and a mutation rate of 0.02. After following the population through 2000 generations, the algorithm converged to a stable solution.

The same search was carried out for models constructed with 1 to 15 variables, and their SEC were used to obtain a prediction residual error sum of squares (PRESS) plot. Let h designate the number of wavelets in the model with the minimum PRESS value. The most parsimonious model was the one with the fewest number of wavelets such that the PRESS for that model was not significantly greater than PRESS for the model with h wavelets (f-test, 99% confidence level).

Class values estimated were either 0 or 1. However, the regression above determined continuous real values. Class separation was determined using values greater that 0.5 as being from class 1 and values less than 0.5 from class 0. For each model, the sensitivity and specificity were determined and used as the criterion for model selection.

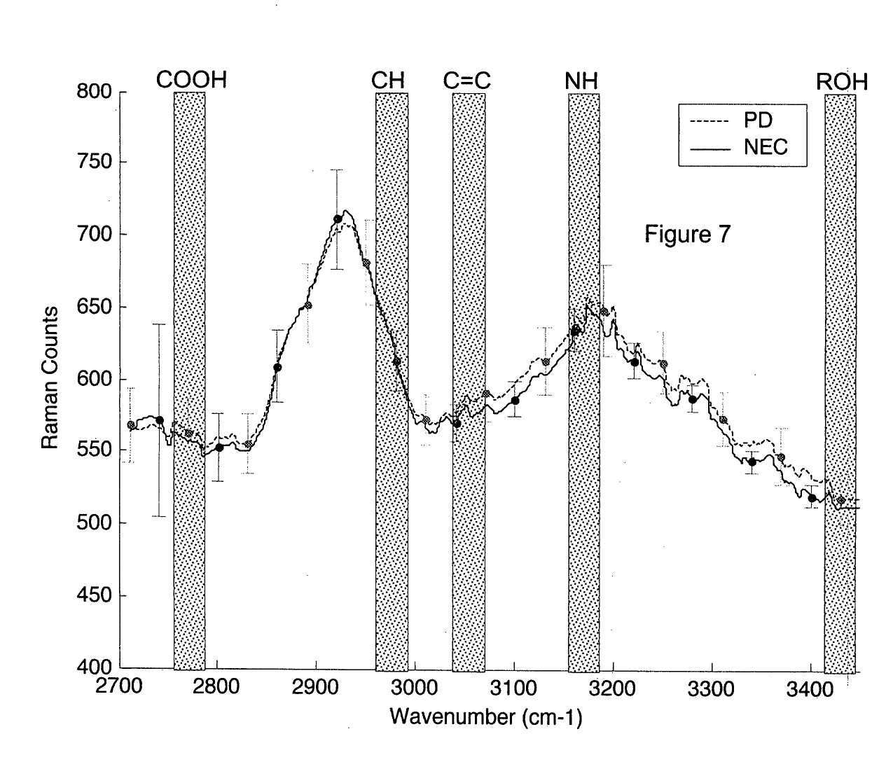

It will be appreciated that the present invention can work well with wavelengths from optical spectra in a variety of ranges, such as the NIR, SWNIR and THz ranges, as would be apparent to a person skilled in the art. In addition to absorption spectra, Raman spectra as illustrated in FIG. 7 may be used. FIG. 7 shows the average differences between NEC and PD. Fluorescence spectra can also be similarly analyzed. In the case of NMR, the analysis technique would be modified, as would be apparent to a person skilled in the art, to identify the desired oxidative stress components and/or perform the correlation with the desired disease or condition.

It will also be appreciated that the present invention can be used to correlate spectra to a disease or condition state, in addition to providing one or more values of oxidative stress. In the latter case, the present invention provides that a processor can generate a value representing a weighted average of a plurality of values for oxidative stress components, such that the weighted average provides a value indicative of a degree of oxidative stress of the patient.

### Application of the Preferred Embodiment

A. Prognostic Use

Through spectroscopic analysis of blood plasma in the SWNIR region a system has been demonstrated permitting rapid diagnosis and distinction between the following disease categories: Alzheimer's Disease Type (AD), Mild Cognitive Impairment (MCI), Vascular Cognitive Impairment (VCI) and Parkinson's disease (PD). The system provides for sensitive, reliable, rapid and inexpensive diagnosis of Alzheimer's Disease.

Based on a hypothesis driven approach, this assignment to AD, MCI, VCI, PD or a normal elderly control (NEC) category was enabled by relating the absorbance at preselected OS-related wavelengths in the SWNIR region to each category. These wavelengths reflect non-specific functional groups of a variety of biomolecules (i.e. oxidative stress components) that, with respect to each category, are believed to be in a different state of oxidation as a result of oxidative stress.

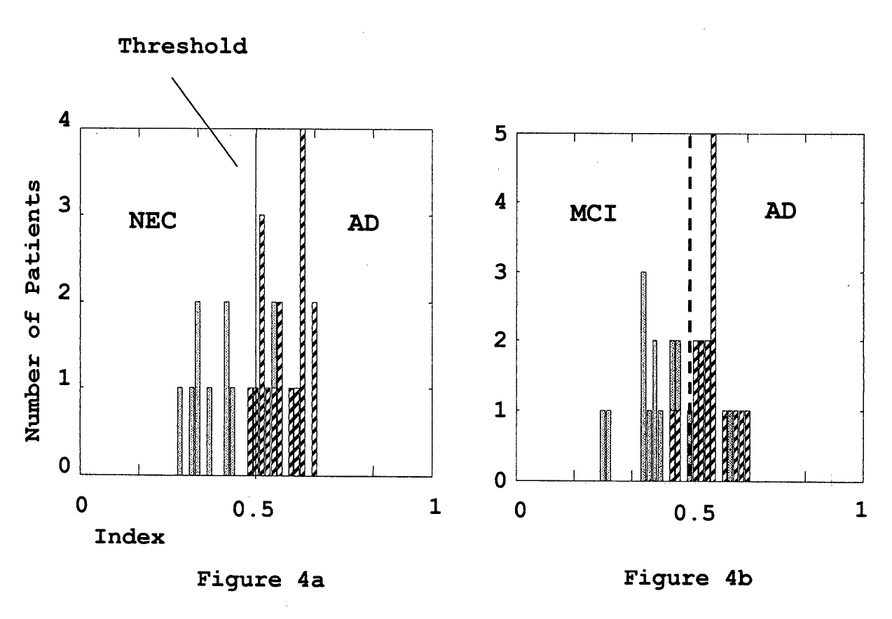

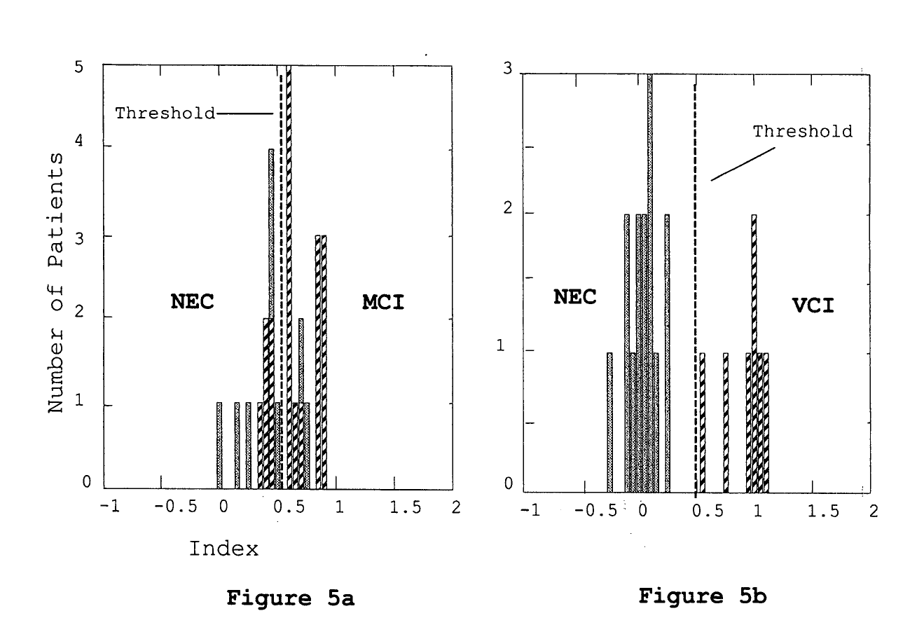

When comparing NEC to AD samples, a sensitivity of 94% and a specificity of 64% were accomplished. Measuring MCI versus AD, a sensitivity of 88% and a specificity of 72% were obtained. These examples are illustrated in FIGS. 4a and 4b. For NEC versus MCI, the results were 72% and 57%, respectively (see FIG. 5a) The numbers rival results published for biological markers in cerebrospinal fluid [Blennow, K., CSF biomarkers for mild cognitive impairment. J Intern Med, 2004. 256(3): p. 224-34; de Leon, M. J., et al., MRI and CSF studies in the early diagnosis of Alzheimer's disease. J Intern Med, 2004. 256(3): p. 205-23]. Importance of this methodology becomes even more evident when emphasizing the simplicity of plasma collection and its relative non-invasiveness as compared to the much more invasive procedure for obtaining and analyzing cerebrospinal fluid.

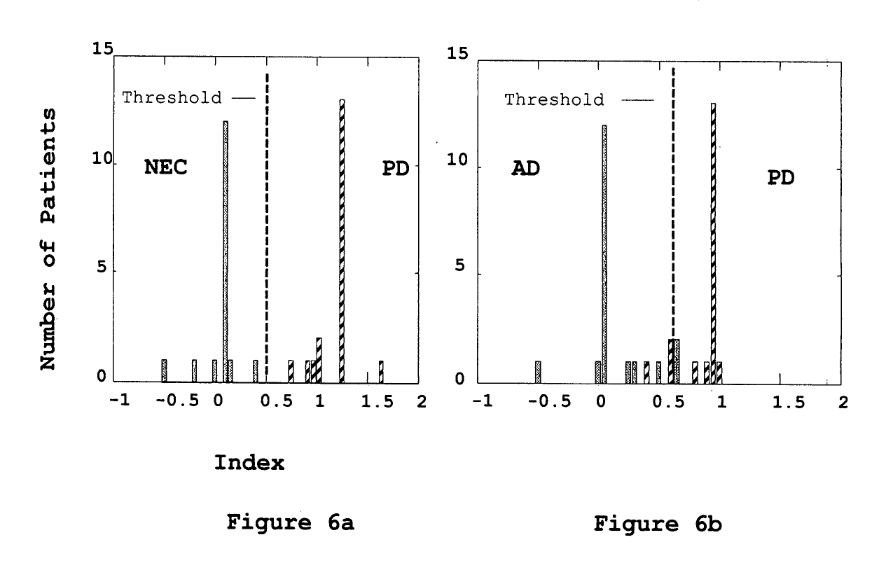

Comparison between any of these three (AD, MCI and NEC) groups and VCI resulted in sensitivities and specificities of 100% (an example is shown in FIG. 5b) thereby possibly extending the applicability of this methodology towards distinction of MCI and AD from Vascular Dementia. With respect to Parkinson's Disease, comparison between NEC and PD yielded both a sensitivity and a specificity of 100% (FIG. 6a). A comparison between AD and PD resulted in a sensitivity of 95% and a specificity of 84% (FIG. 6b).

Possible interference stemming from medication was evaluated for Aricept™, a drug of the acetylcholinesterase inhibitor type prescribed to some patients in the AD group to decelerate progression of Alzheimer's Disease. No correlation could be measured between spectral response and presence of this drug implying a profound robustness of this methodology with respect to this drug (on a side note, this also indicates the possibility of a rather limited effect of this medication with respect to oxidative stress levels).

The analytical procedure was validated with respect to sample cell conditioning and sample preparation. A rinsing procedure was developed to prevent possible adsorption of plasma components to the sample cell walls. Freezing and thawing cycles as well as centrifugation times were standardized since variations of these parameters were found to greatly influence on the spectral response. No dependence of the spectral response on the total storage time at −80° C. was detected. The nature of anticoagulant added to the sample (EDTA versus Heparin) was found to influence the spectral response as well. Therefore, only collectives prepared with the same anticoagulant were considered.

It should also be mentioned that sample and reagent consumption are minimal. The small size of the instrument further permits easy portability.

B. Clinical Use: Alzheirmer's Disease

AD Diagnosis by Short Wavelength Near Infrared (SWNIR) Spectroscopy

As described above, there is ample evidence of oxidative substrate modifications in neural and systemic AD tissues that can be exploited in the development of novel biological markers of this disease. In this specification, we provide evidence that SWNIR spectra rapidly and effectively distinguish between plasma samples derived from early AD, MCI and normal elderly control (NEC) patients based on the differential oxidation of circulating protein constituents in these conditions.

We show that a redox signature satisfies many of the criteria for a useful biological marker in sporadic AD as defined in a Consensus Report sponsored by the Ronald and Nancy Reagan Research Institute (Alzheimer's Association) and National Institute on Aging (Neurobiology of Aging 19:107-167, 1998): 1) SWNIR Spectroscopy exhibits high sensitivity and specificity in differentiating sporadic AD from cognitively-normal elderly controls, and MCI subjects from both NEC and early sporadic AD patients. The test is thus capable of detecting AD in the very earliest phases of the illness and in pre-symptomatic (MCI) individuals. 2) A test based on SWNIR Spectroscopy is relatively non-invasive and inexpensive, and could be readily available in many hospital laboratories. 3) As a biophysical indicator of blood protein oxidation, the SWNIR Spectroscopy-derived redox signature reflects an intrinsic aspect of AD pathophysiology, viz. central and systemic oxidative stress.

A typical patient with memory complaints presents to a family practitioner or is referred to a neurologist or geriatrician for evaluation of the etiology (cause) of the symptoms. The diagnostic evaluation generally consists of (i) a detailed medical, neurological, social and family history, (ii) a general and neurological examination, (iii) a panel of blood tests to exclude metabolic and potentially reversible causes of memory loss and dementia, (iv) referral to a clinical neuropsychologist for formal (quantitative) neuropsychological testing, and (V) referral for a neuroimaging procedure (CT or MRI of the head; occasionally, PET or SPECT scanning). CSF examination is not routinely performed in Canada and the US as part of the dementia evaluation unless highly specific etiologies (e.g. neurosyphilis, HIV encephalitis) are entertained.

SWNIR spectroscopy for the diagnosis of AD can entail the following protocol: (i) In the family physician's office or Memory Clinic, 10 cc venous blood is drawn in an EDTA-anticoagulated tube and sent on ice to the SWNIR spectroscopy laboratory; (ii) The whole blood is layered over a Ficoll density gradient and centrifuged at 1000 g for 20 minutes. The top plasma layer is collected, aliquoted and frozen at −80° C.; (iii) In preparation for SWNIR analysis, the sample is thawed, injected into the spectrometer and SWNIR spectra are taken as described above; and (iv) The spectra are classified as “normal”, “MCI”, “AD”, “VCI/VD” based on the aforementioned algorithms and comparison with reference spectra obtained from well-ascertained patients from each of these diagnostic categories. Spectra not conforming to any of these diagnostic categories would be classified as “other” or “inconclusive”. The laboratory director provides a copy of the patient's spectrum and its interpretation to the referring physician. The latter integrates the SWNIR data with the clinical neuropsychological, biochemical and neuroimaging data to arrive at a likely diagnosis that s/he communicates to the patient and/or the referring physician. Alternatively, instead of drawing blood, blood can be measured transcutaneously.

SWNIR Spectroscopy for Prognostication in MCI:

In addition to providing an important neurodiagnostic for early AD, we show that SWNIR spectroscopy may be particularly useful as a novel prognosticator in subjects with MCI by differentiating MCI patients with abnormal blood NIR spectra at high risk for development of AD from neuropsychologically-identical cases manifesting NIR spectra in the normal range who remain at low risk for conversion to incipient AD. As such, NIR analysis of MCI patients would provide vital prognostic information that could facilitate patient and family counseling, the stratification of sub-groups in the design of clinical drug trials and the interpretation of treatment outcome measures.

There are clinical situations where SWNIR spectroscopy, in combination with one or more additional diagnostic tests, may significantly enhance the accuracy of diagnosing AD over performance of SWNIR spectroscopy or the other diagnostic modality alone. Two examples follow.

(i) Diagnosing AD in a patient with major depression: Patients with depression often complain of memory loss and the latter may constitute an initial symptom that leads the patient to be referred to a neurologist or Memory Clinic for work-up of possible dementia. Due to overlapping symptomatology involving memory function, in general, a new diagnosis of AD cannot be made with any degree of precision until the depressive symptoms have been treated by pharmacological or other means (a process usually requiring a minimum of three weeks). Thereafter, the patient can be re-tested for memory loss and other cognitive dysfunction and a diagnosis of AD, other dementia or normal cognition may be rendered. The advent of a method based on SWNIR spectroscopy that distinguishes AD blood samples from those of non-AD samples, including patients with depression but no AD pathology, would permit immediate rendering of an AD diagnosis (or not) in patients with depression without the necessity of first treating the underlying affective disorder. Similar benefits of SWNIR spectroscopy would accrue in the course of evaluating patients for possible AD with other concomitant conditions that may confound clinical and neuropsychological testing, such as toxic or metabolic encephalopathy (delirium), language disorder (aphasia) or suppressed level of consciousness (stupor, coma).

(ii) Diagnosing AD in patients with chronic inflammatory disorders: As described above, the SWNIR spectra of plasma that distinguish AD are thought to represent aberrant oxidation of specific plasma proteins in the circulation of AD patients. Conceivably, potentially similar patterns of plasma protein oxidation may arise in patients with chronic inflammatory or metabolic disorders characterized by sustained systemic oxidative stress, such as rheumatoid arthritis. The presence of said disorder in a patient under investigation for memory loss may therefore confound the diagnosis of AD rendered solely on the basis of SWNIR spectroscopy. In the latter situation, a second diagnostic modality, such as formal neuropsychological testing, determination of CSF—amyloid and tau concentrations or neuroimaging, would be required in addition to SWNIR spectroscopy to confirm or refute a diagnosis of AD.

C. Drug Efficacy: AD and other OS-related conditions

Although cholinesterase inhibitors are routinely used in clinical practice to ameliorate the symptoms of AD, there is currently no approved medication that unequivocally attenuates neuronal degeneration and disease progression in these patients. On the basis of the data reviewed above, there is considerable interest in developing safe and effective antioxidants to retard or arrest OS-related neuronal damage and attrition in AD. Current monitoring of the efficacy of such interventions is not trivial and requires serial neuropsychological testing and neuroimaging that is labor-intensive, expensive, and often highly-dependent on the cooperation of the test subjects. As an indicator of potential therapeutic efficacy, SWNIR spectroscopy may be used to detect normalization (or not) of aberrant blood spectra in AD patients and in subjects with other OS-related conditions following oral or parenteral administration of a test antioxidant compound.

In such situations, we show that SWNIR spectroscopy would allow objective, non-invasive, rapid, repeated and reproducible monitoring of the drug's antioxidant potential and pharmacokinetics irrespective of the patients' level of consciousness and degree of cognitive/behavioural impairment. Data accruing from these SWNIR-based analyses could be used to rapidly and effectively screen candidate pharmaceuticals for inclusion in subsequent conventional clinical trials.

Oxidative stress (free radical damage) has been implicated in the pathogenesis of numerous neurological and medical disorders. As a result, efforts are currently underway to prevent, ameliorate, arrest or reverse some of these conditions by administration of antioxidants as pharmaceutical agents or dietary supplements. Because monitoring of clinical outcomes of such treatments is generally labor-intensive, costly and subjective, there exists a great need to develop surrogate biological markers of effective therapeutic interventions. There currently exists the capacity to monitor, in quantitative fashion, levels of oxidized blood proteins, lipids and nucleic acids before, during and after antioxidant administration as surrogate markers of potentially effective interventions. However, these biochemical determinations tend to require sophisticated sample preparation and analyses that are expensive, time and labor-intensive, difficult to standardize and restricted to highly specialized laboratories.

SWNIR spectroscopy for detection and measurement of plasma protein oxidation can greatly facilitate clinical and experimental monitoring of antioxidant interventions in said conditions (including AD) because (i) this method, based on SWNIR spectroscopy is a far more rapid methodology for detecting oxidation of plasma constituents than conventional (ELISA, HPLC) methods. Refinement of the method to accommodate in vivo whole blood measurements (akin to oximetry) would permit repeated analyses of drug efficacy and/or a component of pharmacokinetics in real-time.

In AD, for example, SWNIR spectroscopy could be performed on plasma samples or whole blood in vivo prior to, at regular intervals during, and following cessation of orally or intravenously administered antioxidant compounds. Partial or complete normalization of AD-specific spectra resulting from the administration of a candidate antioxidant compound may provide essential data concerning the potency of the medication, the duration of its biochemical effect, and its appropriateness for large-scale, long-term testing as a potential anti-AD drug. (ii) SWNIR spectroscopy is more economical and versatile than existing techniques for monitoring plasma oxidative stress and can be made readily available in all hospitals and diagnostic facilities.

D. ICU Monitoring (‘Redoximetry’)

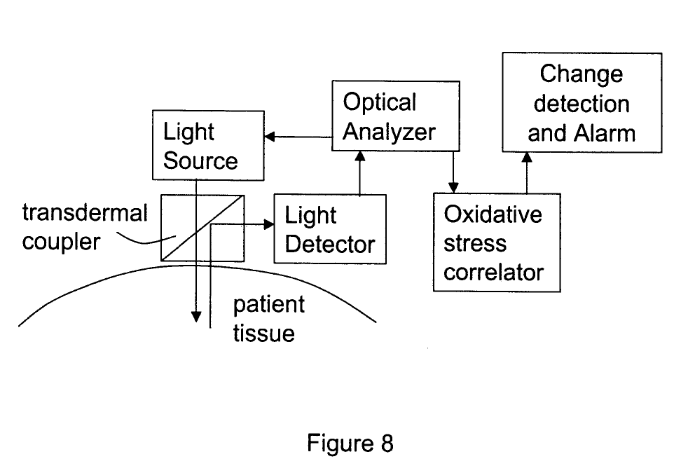

In certain OS-related conditions, like acute systemic bacterial sepsis (Albuszies, G. and U. B. Bruckner, Antioxidant therapy in sepsis. Intensive Care Med, 2003. 29(10): p. 1632-6; Abu-Zidan, F. M., L. D. Plank, and J. A. Windsor, Proteolysis in severe sepsis is related to oxidation of plasma protein. Eur J Surg, 2002. 168(2): p. 119-23], we show that the pattern and extent of abnormalities in the SWNIR spectra can be correlated in real time with the immediate clinical status of the patient. Care for such individuals in an ICU setting can benefit from serial, non-invasive SWNIR spectroscopic measurements of oxidative stress components in blood as a marker of disease severity and efficacy of acute (pharmacological and non-pharmacological) medical interventions. Novel implementation of SWNIR spectroscopy as a real-time ‘redoximeter’ in such patients would be akin to the common use of pulse oximetry to monitor hemoglobin saturation and oxygenation status of acutely-ill patients in an ICU setting, as shown in FIG. 8.

For example, a ‘redoximeter’ monitoring oxidative stress in ICU patients in real-time can be set to trigger an alarm whenever SWNIR spectra corresponding to oxidation of plasma protein constituents shift more than 1-2 standard deviations (to be determined empirically) from normal control values. The SWNIR evidence of augmented oxidative stress may indicate exacerbation of an underlying medical condition (e.g. worsening hyperglycemia in a diabetic), the development of an intercurrent illness (e.g. bacterial sepsis) or an iatrogenic effect (e.g. adverse reaction to medication). The ICU staff may respond to the redoximetry alarm by confirming disease exacerbation or development of concomitant conditions using conventional testing, reviewing ongoing therapeutic regiments, and possibly administration of antioxidant medications. Partial or complete re-normalization of the SWNIR spectra would silence the alarm and provide evident of effective intervention, disease amelioration and stabilization of the patient.

The embodiments of the invention described above are intended to be exemplary only. The scope of the invention is therefor intended to be limited solely by the scope of the appended claims.

1. A method of generating probability data of the presence of an oxidative stress dependent disease, the method comprising:
analyzing a spectrum obtained of the biological fluid using an analytical modality;
generating probability data using the acquired spectrum and a correlation between an oxidative stress dependent disease.

2. The method as claimed in claim 1, wherein said analytical modality is multi-wavelength optical absorption.

3. The method as claimed in claim 1, wherein said analytical modality is one of optical Raman scattering and optical fluorescence.

4. The method as claimed in claim 2, wherein said optical spectrum is short wavelength near infrared.

5. The method as claimed in claim 2, wherein said optical spectrum is near infrared.

6. The method as claimed in claim 2 or 3, wherein said optical spectrum is in the THz range.

7. The method as claimed in claim 2, wherein said chosen analytical modality is NMR.

8. The method as claimed in claim 1, wherein said disease is Alzheimer's disease (AD).

9. The method as claimed in claim 1, wherein said disease is mild cognitive impairment (MCI).

10. The method as claimed in claim 1, wherein said disease is Parkinson's disease (PD).

11. The method as claimed in claim 1, wherein said disease is vascular cognitive impairment (VCI).

12. A method of clinical diagnosis, the method comprising:
obtaining a redox signature of oxidative stress components in a biological fluid by analyzing a spectrum obtained of the biological fluid using an analytical modality and generating probability data using the acquired spectrum and a correlation between an oxidative stress dependent disease;
observing at least one additional clinical condition; and
obtaining a diagnosis using said at least one additional condition and said redox signature, wherein said diagnosis is not enabled by only one of said measurement and said at least one additional condition.

13. A method of studying efficacy of a drug or treatment intended to treat an oxidative stress related disease, the method comprising:
determining over time a redox signature by analyzing a spectrum obtained of the biological fluid using an analytical modality and generating probability data using the acquired spectrum and a correlation between an oxidative stress dependent disease pursuant to said drug or treatment.

14. A method of monitoring oxidative stress component, the method comprising:
continuously or frequently determining, at least one oxidative stress component by analyzing a spectrum obtained of the biological fluid using an analytical modality and generating probability data using the acquired spectrum and a correlation between an oxidative stress dependent disease; and
detecting a change in said one or more oxidative stress component.

15. The method as claimed in claim 14, wherein a value representing a weighted average of a plurality of said oxidative stress components is calculated, and said detecting a change comprises detecting a change in said average to detect a change in a degree of oxidative stress.

16. The method as claimed in claim 14, further comprising triggering an alarm in response to said detecting a change.

17. An apparatus for measuring at least one oxidative stress component in a patient comprising:
an optical analyzer having a light source and a light detector for measuring at least one optical property of a medium and generating optical measurement data;
a transdermal coupler for coupling the optical analyzer with biological fluid of the patient; and
a processor analyzing the optical measurement data and generating a value for one or more oxidative stress component in the form of a redox signature for the patient.

18. The apparatus as claimed in claim 17, wherein said processor generates value representing a weighted average of a plurality of said values for oxidative stress components, wherein said average provides a value indicative of a degree of oxidative stress of the patient.

19. The apparatus as claimed in claim 17, wherein said optical analyzer measures multi-wavelength optical absorption.

20. The apparatus as claimed in claim 17, wherein said optical analyzer measures one of optical Raman scattering and optical fluorescence.

21. The apparatus as claimed in claim 19, wherein said light source emits short wavelength near infrared.

22. The apparatus as claimed in claim 19, wherein said light source emits near infrared light.

23. The apparatus as claimed in claim 19, wherein said light source emits light in the THz range.

24. The apparatus as claimed in claim 19, wherein said light source is a radio frequency source.

25. The apparatus as claimed in claim 17, wherein said processor detects a change in said one or more oxidative stress component, further comprising an alarm module for generating an alarm signal in response to said change.
